(19) United States
(12) Patent Application Publication
(10) Pub. No.: US 2013/0338237 A1
(43) Pub. Date: Dec. 19, 2013

(54) PREPARATION OF BRUSHITE AND OCTACALCIUM PHOSPHATE GRANULES

(71) Applicant: Ahmet Cuneyt Tas, Ankara (TR)

(72) Inventor: Ahmet Cuneyt Tas, Ankara (TR)

(21) Appl. No.: 13/993,253

(22) PCT Filed: Dec. 31, 2010
(86) PCT No.: PCT/EP2010/070957

§ 371 Date: Jun. 11, 2013

(30) Foreign Application Priority Data
Mar. 22, 2011 (US) 61466381
Sep. 30, 2011 (US) 61542035

(51) Int. Cl. A61K 47/02; C01B 25/32

(52) U.S. Cl. CPC A61K 47/02; C01B 25/324; C01B 25/322
USPC 514/770; 423/309

(57) ABSTRACT

Brushite (DCPD, dicalcium phosphate dihydrate, CaHPO₄-2HH₂O) and octacaicium phosphate (OCP, Ca₈(HPO₄)₂(PO₄)₄-5H₂O) granules in the millimetre size range were prepared by using calcium carbonate granules of marble-origin as the starting material. The method of the invention comprised of soaking the marble granules in aqueous solutions containing phosphate and/or calcium ions at temperatures between 20° and 75° C. The load-bearing DCPD and OCP granules of this invention are useful in maxillofacial and orthopedic void/bone defect filiing and grafting applications.

### TECHNICAL FIELD

This invention relates to the preparation of load-bearing granules of brushite (DCPD, dicalcium phosphate dihydrate. CaHPO4.2H2O) or octacaicium phosphate (OCP, Ca8(HPO4)2(PO4)4.5H2O) with sizes in the millimeter range. Although the powders of brushite and octacalcium phosphate can be prepared rather easily by a person skilled in the art, their granules can not be prepared by the common techniques based on, for instance, blending/consolidating powders of DCPD or OCP with a polymer, followed by high temperature treatment/calcination, mainly because DCPD and OCP can not withstand temperatures higher than 80° C., they will simply decompose into monetite (DCPA, CaHPO4) or hydroxyapatite (HA, Ca10(PO4)6(OH)2), respectively, The approach taken in this invention was to use mechanically strong marble (calcium carbonate) granules as the template and transforming them into either DCPD or OCP by immersion in specially prepared solutions at temperatures between 20° and 75° C. The sizes of the produced DCPD and OCP granules imitated the initial sizes (0,5-4 mm) of the marble granules used.

### PRIOR ART

The solubility behavior of biominerals is important for both their formation and disappearance in biological environments, such as human bones. The solubility of biominerals depends on their composition and crystallographic structure. The logarithm of the thermodynamic solubility of calcium carbonate (CaCO₃) and dicalcium phosphate dihydrate (brushite, CaHPO₄.2H₂O) are numerically close to one another; log K_{SP} for CaCO₃ is −8.55, whereas for CaHPO₄.2H₂O it is −6.60. On the other hand, the log K_{SP} of HA (hydroxyapatite, Ca₁₀(PO₄)₆(OH)₂) is −117.1. Hydroxyapatite has the lowest solubility among all the Ca-rich phases of the Ca—P—O—H system. The log K_{SP} value for OCP (octacalcium phosphate, Ca₈(HPO₄)₂(PO₄)₄.5H₂O) is equal to −72.5. (1)

When a patient is in need of a bone substitute material in amounts much beyond that of the surgeon can extract from the pelvis of the same patient (i.e., autologous bone chips), synthetic bone graft or bone substitute materials come into play as a good choice.

Clinically-successful synthetic bone substitute materials are usually required to (i) exhibit a high degree of in vivo resorbability to actively take part in the bone remodeling processes, (ii) be partially resorbed by the osteoclast cells, and (iii) simultaneously allow the proliferation of osteoblast cells on their surfaces. Synthetic HA, owing to its very low solubility, does not display any in vivo resorbability, and in most cases bone substitutes made out of synthetic HA act like a cemetery for the eroding osteoclast cells. Synthetic HA only allows the proliferation of osteoblast cells and bone growth on its surfaces (i.e., osteoconductivity). This has been why, especially over the last decade, bone substitutes based on brushite, instead of HA, gained increasing popularity (2-10).

According to the current state-of-the-art, brushite can be synthesized in (a) powder form and (b) cement form. Brushite powders can be readily synthesized at room temperature by the rapid addition of a solution of CaCl₂.2H₂O (or Ca(NO₃)₂.4H₂O or Ca(CH₃COO)₂.H₂O) to another solution of (NH₄)₂HPO₄ (or Na₂HPO₄.) at the nominal Ca/P molar ratio in the resultant solution mixture to be adjusted to around 1.0, followed by stirring for less than an hour and finally by filtering the precipitated crystals of brushite out of the mother liquor. Synthesis of brushite powders is easy and reproducible (11). On the other hand, the production of brushite cements is commonly achieved by reacting β-tricalcium phosphate (β-TCP, β-Ca₃(PO₄)₂) powders either with a solution of H₃PO₄ (l) or Ca(H₂PO₄)₂ (s) (2-10). However, brushite granules were only mentioned in the previous art as a by-product of the brushite cements. First, a brushite cement was produced by reacting β-TCP with either H₃PO₄ Ca(H₂PO₄)₂, and then the set brushite cement was crushed (via milling) into granular form, and later this brushite was converted into rnonetite (CaHPO4 by heating the brushite) (12-14). In the brushite cements, there is one major disadvantage though; that is, the reaction is never complete and the cores of the granules will always contain unreacted β-TCP at about 10 to 25 wt %. In other words, the formed brushite cement or granules would only be 75 to 90% pure, at the best. When the cores of those monetiteibrushite granules were comprised of β-TCP, this automatically means that the cores of those granules were made of a material of quite lower solubility, i.e., log K_{SP} of β-TCP is −81.7 (15), This is an order of magnitude lower solubility in comparison to that of brushite.

The preparation of brush e granules from calcium carbonateimarble granules is yet unheard of. Unfortunately, only a few US patents (16-18) focused on converting high solubility brushite into low solubility hydroxyapatite, which therefore would significantly decrease their ability to take part in bone remodeling processes.

The synthesis of OCR powders is not as easy as those brushite powders; however. OCR powder synthesis is well-documented (19-31). It is not usually possible to synthesize OCP powders at room temperature as readily as the brushite powders. For instance, calcium acetate and carboxylate ions were falsely believed to be essential in synthesizing OCP powders (20-24). Acetate or carboxylate ions are not necessarily needed to crystallize OCP in aqueous solutions. One of the simpler methods of ociacalcium phosphate powder synthesis was disclosed by Ban and Hasegawa (32), and in that study they mixed an aqueous suspension containing CaCO₃ powders with either CaHPO₄ or CaHPO₄.2H₂O powders, followed by stirring the suspension at 35-68° C. for 5 to 50 hours. This method showed that it was possible to react CaCO₃ and CaHPO₄.2H₂O in an aqueous solution to synthesize the powders of OCP.

The preparation of octacalcium phosphate granules with a load-bearing ability starting with brushite granules was still unprecedented. Powders of OCR is almost useless.

### OBJECT OF THE INVENTION

It is an object of this invention to produce brushite (CaHPO₄.2H₂O) granules of load-bearing ability by starting with commercially available calcium carbonate/marble granules between the sizes of 1 to 2 mm, by statically reacting those in an aqueous (starting with doubly distilled water), transparent and HPO₄²⁻/H₂PO₄⁻ ion-containing solution of pH 4.0 to 4.2, at room temperature (20±1° C.).

It is also an object of this invention to produce octacalcium phosphate (Ca8(HPO₄)₂(PO₄)₄.5H₂O) granules of load-bearing ability by starting with the above-mentioned brushite granules in the size range of 1 to 2 mm, while using an aqueous, transparent, precipitate-free and Tris-HCl buffered solution (of pH 7.4) containing in it the dissolved inorganic salts of NaCl, KCl, Na₂HPO4, and CaCl₂.2H₂O, in proper amounts, at 37-75° C.

### BRIEF DESCRIPTION OF THE DRAWINGS

FIG. 1; Scanning electron microscope (SEM) photograph of the starting marble granules

FIG. 2 A close-up optical photograph of the brushite granules

FIG. 3a Low-mag SEM photograph of brushite granules

FIG. 3b High-mag SEM photograph of brushite granules

FIG. 4 X-ray diffraction (XRD) identification of obtained brushite granules

FIG. 5a Low-mag SEM photograph of octacalciurn phosphate granules

FIG. 5b High-mag SEM photograph of octacalcium phosphate granules

FIG. 6 XRD identification of the obtained octacaicium phosphate granules

### DETAILED DESCRIPTION OF THE INVENTION

This invention uses marble (of the pure calcite. CaCO₃, form) granules with sizes 1 to 2 mm as the starting material or template, Firstly, the method of this invention envisages the production of brushite granules by starting with the marble granules. Secondly and separately, the method of this invention comprises the production of octacalcium granules by starting with the brushite granules produced by this invention.

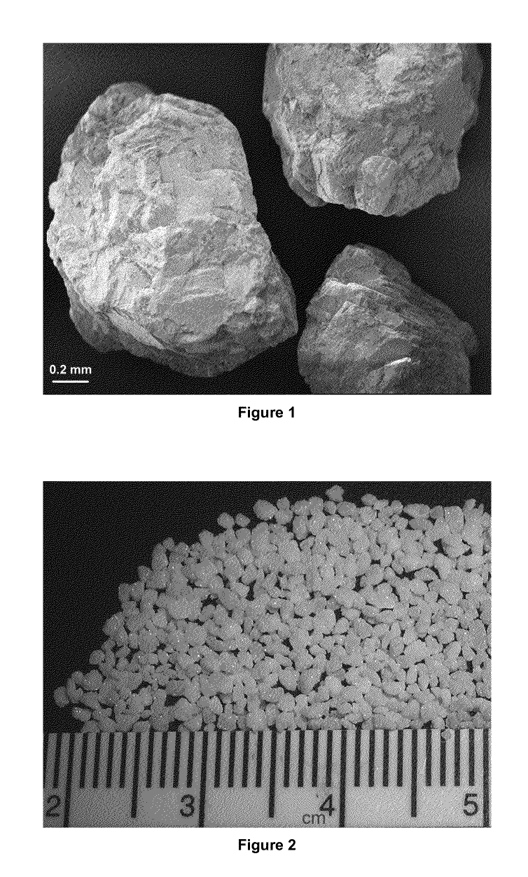

Marble samples used were commercially available (Merck KGaA, Darmstadt, Germany, Catalog No: 1.05986.1000). The chemical analyses of these marble granules were performed by using ICP-OES throughout this study, The marble granules were found to consist of 55.5% CaO, 0.2% MgO, <0.1%SiO₂, and <0.1% Fe₂O₃. X-ray diffraction (XRD) and Fourier-transform infrared spectroscopy (FTIR) analyses of the marble granules showed that they were consisting of single-phase calcite, and for the former of these analyses the collected data were conforming very well with the powder diffraction file (PDF) of 5-0586 of International Centre for Diffraction Data (ICDD). Above-mentioned marble granules were used as-received, without any further purification or chemicailphysicai treatment whatsoever. A typical scanning electron microscope (SEM) photograph of the starting marble granules was shown in FIG. 1.

This invention do not aim at producing hydroxyapatite granules since this phase has a very low in vitro solubility and since it is a bioceramic that cannot easily take part in bone remodeling or bone turnover processes, in vivo.

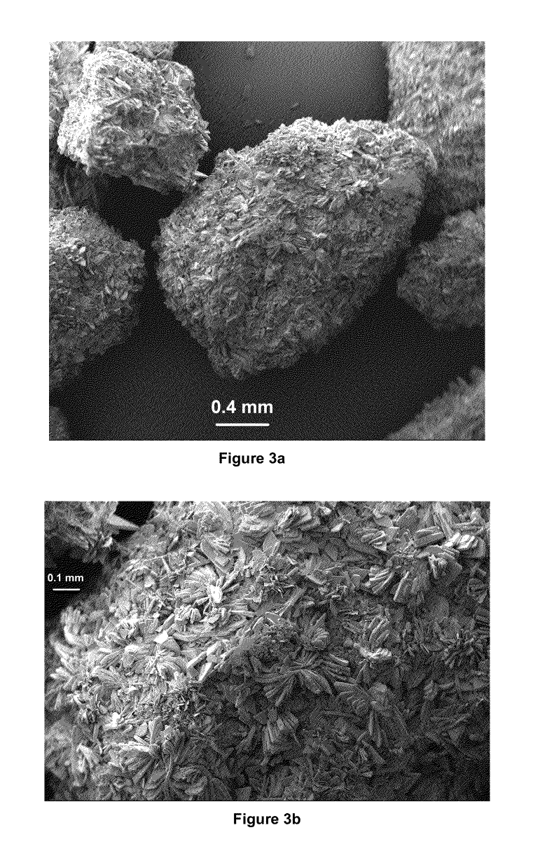

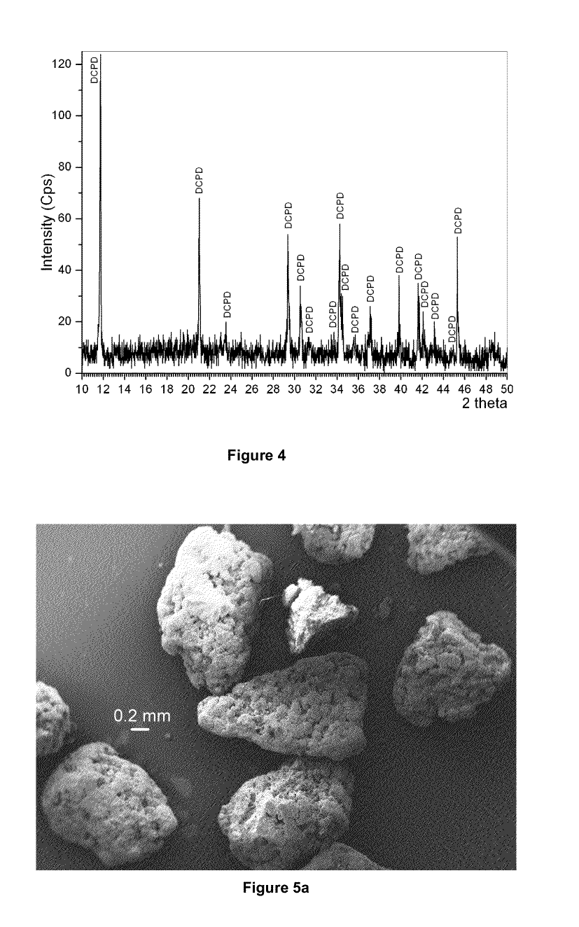

The solutions developed for transforming the calcium carbonate/marble granules were quite easy to prepare; they were comprised of either NH₄H₂PO₄ or NaH₂PO₄ or KH₂PO₄ (or an appropriate mixture of those) dissolved, over a certain concentration range, in doubly distilled water. The pH values of these solutions were adjusted over the range of 4.0 to 4.2 at room temperature. Similarly, solutions of concentrated ortho-phosphoric acid (H₃PO₄) whose pH values were raised to around 4 by slow additions of the appropriate amounts of NH₄OH (in liquid form) or NaOH (either in liquid or solid pellet form), were also prepared and successfully used in the production of brushite granules from the starting calcium carbonate/marble granules. The pre-weighed amounts of marble granules were placed into clean glass media bottles, followed by adding one of the above-mentioned solutions into the specific bottle. Once the solution and the granules were wetted one another, the glass bottle was tightly capped and set aside, at room temperature, and there was no need to stir the granules during the entire process. The granules were kept at room temperature in these solutions from 6 to 24 hours, and at the end of the prescribed period of immersion, granules were filtered off, washed with ample amounts of doubly distilled water, and finally dried in clean glass watch glasses in a microprocessor-controlled drying oven at 37° C., overnight. There was absolutely no need to increase the temperature (from room temperature) to form the brushite granules. The obtained granules were characterized by using optical microscopy, scanning electron microscopy. X-ray diffraction and Fourier-transform infrared spectroscopy. The extremely shiny brushite crystals on the produced granules were easily visible through an optical microscope, and they were best identified by using a scanning electron microscope. A close-up optical photograph of the brushite granules was shown in FIG. 2. The shining visible in the brushite granules (in FIG. 2) were due to the brushite crystals depicted in FIG. 3. A couple of characteristic scanning electron microscope photomigraphs of the brushite granules were shown in FIG. 3. FIG. 4 depicted the X-ray diffraction (XRD) data of the produced brushite granules, which conformed very closely to that of ICDD-PDF 9-0077.

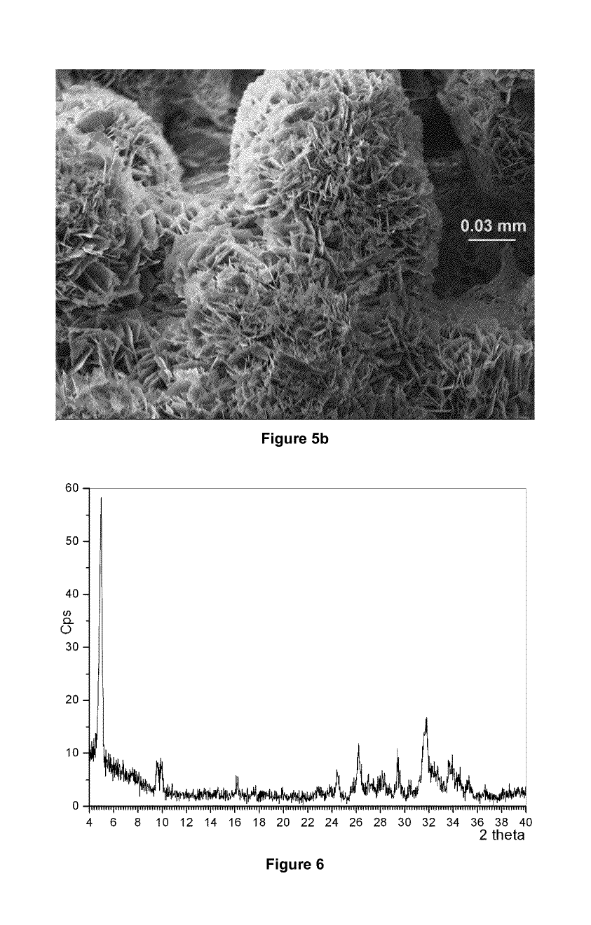

For the production of octacalcium phosphate (OCP) granules, the easiest method was to soak the above-mentioned brushite granules in specially-prepared aqueous solutions. Brushite granules transformed into octacalciurn phosphate granules in such solutions without a difficulty. The solutions used to form OCP granules were prepared by dissolving NaCl, KCl, Na₂HPO₄ and CaCl₂.2H₂O, followed by adjusting the solution pH at the physiological blood plasma pH of 7.4 by using Tris-HCl pair. Tris-HCl use could also be avoided if one added proper amounts of NaHCO₃ into the above-mentioned solutions and replaced Na₂HPO₄ with NaH₂PO₄2H₂O, and by this way it would also be possible to obtain transparent, precipitate-free solutions of pH around 7.4, The brushite granules were statically (i.e., without stirring) soaked in these solutions, in tightly-capped glass media bottles at 37°-75° C., from 24 to 168 hours, time required to form OCP granules strongly depending on the temperature employed, Increasing the soaking temperature to above 37° C. drastically decreased the immersion time (towards 24 h). The morphology of the OCP granules was shown in the SEM photomicrographs of FIGS. 5a and 5b. The characteristic OCP nano-platelets (interlocking and intermingling with one another) were especially visible in the high-mag SEM micrograph of FIG. 5b. The XRD data collected on these granules, shown in FIG. 6, proved beyond doubt that the granules were indeed comprised of OCP phase, the peak positions and intensities of the data conforming well to that of the ICDD PDF 26-1056.

The conversion of brushite granules into octacalcium phosphate granules most probably took place according to the below reaction:

6 CaHPO₄.2H₂O(s)+2 Ca²⁺(aq) Ca₆(HPO₄)₂(PO₄)₄.5H₂O(s)+7 H₂O(aq)+4 H⁺(aq)

The above reaction also explains why one observes a slight decrease in solution pH (which was initially at 7.4) to about 6.6 to 6.8.

If one ever wonders about the possibility of finding small amounts of unreacted calcium carbonate at the very cores of both brushite and octacalcium phosphate granules, then it should be noted that the thermodynamic solubility (i.e., log K_{SP}) of calcium carbonate would still be “order of magnitudes higher” than those of β-TCP and hydroxyapatite, and osteoclast cells erode calcium carbonate, in direct comparison to both of these phases, very easily (33). Resorbability of the produced granules is the main concern of this invention. Surgeons may find such granules extremely versatile and useful since they could be formed into plugs (for bony defect/void filling applications) by using blood clotting behavior, or such granules can be impregnated with growth factors, platelet-rich plasma and alike to be extracted from the patient's own blood. The load-bearing ability of the granules of this invention (with a compressive strength at around 250 kg/cm²) will not cause the granules to crumble between the fingers of the surgeons, as many of the previous granules do.

### WORKING EXAMPLES

### Working Example-1

### Preparation of Brushite Granules

10 grams of NH₄H₂PO₄ is dissolved in 50 mL of doubly distilled water. The pH of this solution is 4. The solution was prepared in a 100 mL-capacity glass media bottle. 2 grams of calcium carbonate/marble granules were placed into the above solution. The solution and the granules in it were not stirred and kept statically at room temperature for about 24 h. At the end of this period the filtered granules were washed with 1 liter of distilled water and dried at 37° C. An upscaling of the phosphate solution volume to 1000 mL and the starting granule weight to 40 grams worked equally well, and the brushite granules obtained from both runs were virtually indistinguishable from one another by electron microscopy, X-ray diffraction and Fourier-transform infrared spectroscopy.

In the above preparation recipe, 10 grams of NH₄H₂PO₄ (equal to 0.0869 moles of H₂PO₄⁻ in 50 mL distilled water can be replaced with
(i) 10.426 grams of NaH₂PO₄ (equal to 0.0869 moles of H₂PO₄⁻) or
(ii) 11.826 grams of KH₂PO₄ (equal to 0.0869 moles of H₂PO₄⁻)
   without causing any noticeable changes in the physico-chemical properties of the produced brushite granules.

Alternatively, to prepare a new solution similar to the above, one can add 120 mL of concentrated H₃PO₄ to 730 mL of doubly distilled water, followed by dropwise addition of 137 mL of concentrated NKOH; the final solution thus obtained will have a pH value equal to 4. 1 mL of the solution will contain 1.889×10⁻³ mole of phosphor in it. This solution can be used equally well with the calcium carbonate/marble granules to produce brushite granules. In using this alternative solution, 3 grams of marble granules will be placed, again in a glass media bottle, into 69 mL of the above solution and will be kept unstirred for about 24 h to form the brushite granules.

### Working Example-2

### Preparation of Octacalcium Phosphate Granules

The solutions shown in Table 1, which were always prepared on a 1000 mL total volume basis, can all be equally well used in producing octacalcium phosphate granules, by starting with the brushite granules synthesized according to the conditions/parameters of Example-1. The solutions shown in Table 1 were prepared by using NaCl. KCl, CaCl₂.2H₂O, MgCl₂.6H₂O, Na₂HPO₄ (in Solutions 1 and 2), NaH2PO₄.2H₂O (in Solution 3), and Tris; unless otherwise noted.

TABLE 1

| | Solutions used in OCP granule production | | | | | | | | | | |
|---|---|---|---|---|---|---|---|---|---|---|---|
| Conc. (mM) | Na⁺ | K⁺ | Mg²⁺ | Ca²⁺ | HPO₄²⁻ | H₂PO₄⁻ | HCO₃⁻ | Cl⁻ | Tris | 1M HCl | pH |
| Solution-1 | 146 | 5 | — | 3.333 | 2 | — | — | 154 | 6.77 g | 55 mL | 7.4 |
| Solution-2 | 145 | 5 | — | 5 | 3 | — | — | 154 | 6.77 g | 55 mL | 7.4 |
| Solution-3 | 127 | 5 | 0.8 | 1.8 | — | 0.9 | 44 | 92 | — | — | 7.5 |

Solution-1 was prepared by adding, one by one, NaCl (8.299 g), KCl (0.373 g), CaCl₂.2H₂O (0.490 g), Na₂HPO₄ (0,284 g) and Tris (6.770 g) into 1000 mL of doubly distilled water in a 1000 mL-capacity glass media bottle at room temperature. 55 of 1 M HCl solution was added dropwise to obtain a transparent solution and finalize the pH at around 7.4.

Similarly, solution-2 was prepared by adding, one by one, NaCl (8.124 g), KCl (0.373 g), CaCl₂.2H₂O (0.735 g), Na₂HPO₄ (0.426 g) and Tris (6.770 g) into 1000 mL. of doubly distilled water in a 1000 mL-capacity glass media bottle at room temperature. 55 mL. of 1 M HCl solution was finally added dropwise to obtain a transparent solution and finalize the pH at around 7.4.

Solution-1 and Solution-2 had the same Ca/P molar ratio of 1.6667. This is mainly because of the fact that in starting the immersion of brushite (for which Ca/P molar ratio=1.0) granules in these solutions, in order to transform brushite into octacalcium phosphate one would need a Ca/P molar ratio greater than 1.33 in the solution side. H

Solution-3 was again prepared by adding, one by one, NaCl (4.792 g), KCl (0.373 g), MgCl₂.6H₂O (0.163 g), NaHCO₃ (3.696 g), CaCl₂.2H₂O (0.265 g), and NaH₂PO₄.2H₂O (0.141 g) into 1000 mL of doubly distilled water in a 1000 mL-capacity glass media bottle at room temperature.

It must be noted that Solution-3 had a Ca/P molar ratio very close to 2 and it contains Mg²⁺ ions, which are known for their ability in slowing the rate of apatitic calcium phosphate formation. Solution-3 is capable of reaching higher temperatures (such as 50° to 75° C.) and convert brushite granules into octacalcium granules much faster than solutions 1 and 2 can.

To summarize, solutions-1 and -2 shall be used to transform the brushite granules of Example-1 into OCP granules at 37° C. over an immersion period of 5 to 7 days, without stirring during that entire period. The solutions can be refreshed, with unused solutions, at every 36 hours interval. Solution-3 can be used to transform the brushite granules of Example-1 into OCP granules at 75° C. in about 24 hours, without a need for solution replenishment.

75 mL aliquots of solutions-1 and -2 were first placed into 100 mL-capacity glass media bottles and 0.35 grams of brushite granules were added into the bottles before starting the “1 week-at-37° C. immersion” runs, without stirring.

To upscale, the solutions-1 and -2 can be placed in 250 mL or 500 mL volumes respectively into 250 mL- or 500 mL-capacity glass media bottles, followed by adding 1.1 or 2.2 grams of brushite granules into those bottles, prior to the start of the “1 week-at-37° C. immersion” runs, without stirring.

A 500 mL portion of solution-3, on the other hand, can be placed into a 500 mL-capacity glass media bottle together with 2 grams of brushite granules of Example-1 and the tightly capped glass bottle were heated at 75° C. in a microprocessor-controlled oven for about 24 hours, without stirring.

At the end of the immersion periods, granules were washed with 1 liter of distilled water and dried at 37° C.

### Working Example-3

### Preparation of Biphasic Brushite-Octaca Phosphate Granules

Since the brushite (DCPD) and octacalcium phosphate (OCR) granules follow in the entire process the size and shape range (or distribution) of the starting calcium carbonate/marble granules and since they do not change their sizes and physical shapes during the process, it will be extremely easy to prepare biphasic, physical mixtures of the two kinds of granules, i.e., by separately weighing the DCPD and OCP granules and than by blending them together. It is thus possible to prepare biphasic mixtures of DCPD and OCR granules, for the first time, according to the below scheme:
10 wt % DCPD—90 wt % OCP,
20 wt % DCPD—80 wt % OCP,
30 wt % DCPD—70 wt % OCP,
40 wt % DCPD—60 wt % OCP
50 wt % DCPD—50 wt % OCP,
60 wt % DCPD—40 wt % OCP,
70 wt % DCPD—30 wt % OCP,
80 wt % DCPD—20 wt % OCP,
90 wt % DCPD—10 wt % OCP.

It must be noted here that the log K_{SP} of DCPD is −6.60, whereas that of OCR is −72.5. The above-mentioned biphasic granule mixtures therefore provide a fine-tuning level of control in terms of the expected solubility/resorbability of such granules and their biphasic physical mixtures in vivo.

### REFERENCES CITED IN THE DESCRIPTION

(1) F. C. M. Driessens and R. M. H. Verbeeck, Biominerals, p, 38, CRC Press, Boca Raton, Fla., 1990.
(2) M. Bohner, H. P, Morkle, P. van Landuyt, G. Trophardy, and J, Lemaitre, “Effect of Several Additives and Their Admixtures on the Physico-chemical Properties of a Calcium Phosphate Cement,” J. Mater. Sci, Mater. M., 11, 111-116 (2000).
(3) B. Flautre, C. Maynou, J. Lemaitre, P. van Landuyt, and R Harclouin, “Bone Colonization of β-TCP Granules Incorporated in Brushite Cements,” J. Biomed, Mater. Res., 63B, 413-417 (2002).
(4) M. Bohner, F. Theiss, D. Apelt, W. Hirsiger, R. Houriet, a Rizzoli, E. Gnos, a Frei, J. A. Auer. and B. von Rechenberg, “Compositional Changes of a Dicalcium Phosphate Dihydrate Cement after Implantation in Sheep,” Biomaterials, 24, 3463-3474 (2003).
(5) D. Apeit, F. Theiss, A. O. El-Warrak, K. Zlinszky, R. Bettschart-Wolfisberger, M. Bohner, S. Matter, J. A. Auer, and B. Von Rechenberg, “in Vivo Behavior of Three Different Injectable Hydraulic Calcium Phosphate Cements,” Biomaterials, 25, 1439-1451 (2004).
(6) M. Bohner and S. Matter, “Brushite Hydraulic Cement Stabilized with a Magnesium Salt,” U.S. Pat. No. 6,733,582 May 11, 2004.
(7) Z. Xia, L. M. Grover, Y. Huang, I. E. Adar)opoulos, U. Gbureck, J. T. Triffitt, R. M. Shelton, and J. E Barralet, “In Vitro Biodegradation of Three Brushite Calcium Phosphate Cements by a Macrophage Cell-line,” Biomaterials, 27, 4557-4565 (2006).
(8) M. P. Hofmann, S. N. Nazhat, U. Gbureck, and J. E. Barralet, “Real-time Monitoring of the Setting Reaction of Brushite-forming Cement using Isothermal Differential Scanning calorimetry,” J. Biomed. Mater. Res., 79B, 360-364 (2006).
(9) D. L. Nihouannen, A. Hacking, U. Gbureck, V. Komarova, and J. E. Barralet, “The Use of RANKL-coated Brushite Cement to Stimulate Bone Remodeiing,” Biomaterials, 29, 3253-3259 (2008).
(10) M. P. Hofmann, A. R. Mohammed, Y. Perrie, U. Gbureck, and J. E. Barralet, “High-strength Resorbable. Brushite Bone Cement with Controlled Drug-releasing Capabilities,” Acta Biomater., 5, 43-49 (2009).
(11) A. C. Tas and S. B. Bhaduri, “Chemical Processing of CaHPO₄.2H₂O: Its Conversion to Hydroxyapatite,” J. Am. Ceram. Soc., 87, 2195-2200 (2004).
(12) F. T. Marino, J. Torres, I. Tresguerrres, L. B. Jerez, and E. L. Cabarcos, “Vertical Bone Augmentation with Granulated Brushite Cement Set in Glycolic Acid,” J. Biomed. Mater. Res., 81A, 93-102 (2007).
(13) F. Tamirni, J. Torres, C. Kathan, R. Baca, C. Clemente, L. Blanco, and E. L. Cabarcos, “Bone Regeneration in Rabbit Calvaria with Novel Monetite Granules,” J. Biomed. Mater. Res., 87A, 980-985 (2008).
(14) F. Tamimi, E. L. Cabarcos, and R. Rubio, “Monetite Matrices and Their Application in Bone Regeneration,” PCT patent No: WO 2009/077210 A1, Jun. 25, 2009.
(15) A. C. Tas, “Monetite (CaHP0.₄) Synthesis in Ethanol at Room Temperature,” J. Am. Ceram. Soc., 92, 2907-2912 (2009).
(16) J. Godber and L. Leite, “Hydroxyapatite Calcium Phosphates, Their Methods of Preparation and Their Applications,” U.S. Pat. No. 7,468,172, Dec. 23, 2008.
(17) J. Godber and L. Leite, “Hydroxyapatite Calcium Phosphates, Their Methods of Preparation and Their Applications,” U.S. Pat. No. 7,767,300, Aug. 3, 2010.
(18) A. Lynn, R. Cameron, S. Best, and W. Bonfield, “Composite Biornaterials Comprising Calcium Phosphate Materials, Collagen and Glycosaminoglycans,” U.S. Pat. No. 7,780,994 Aug. 24, 2010.
(19) M. lijima, “Formation of Octacalcium Phosphate in vitro,” in Octacalcium Phosphate, L.C. Chow and E. D. Eanes (Eds,), Monogr, Oral Sci., Basel, Switzerland, Karger Press, 2001, Vol. 18, pp. 17-49.
(20) R. Z. LeGeros, “Preparation of Octacalcium Phosphate (OCP): A Direct Fast Method,” Calcif. Tissue Int., 37, 194-197 (1985).
(21) R. Z. LeGeros, Daculsi, I. Orly, T. Abergas, and W. Torres, “Solution-mediated Transformation of Octacalcium Phosphate (OCP) to Apatite,” Scanning Microscopy, 3, 129-138 (1989),
(22) M. Markovic, B. O. Fowler, and W. E. Brown, “Ociacalciurn Phosphate Carboxylates. 1. Preparation and Identification,” Chem. Mater., 5, 1401-1405 (1993).
(23) M. Markovic, B. O. Fowler, and W. E. Brown, “Octacalcium Phosphate Carboxylates. 2. Characterization and Structural Considerations,” Chem. Mater., 5, 1406-1416 (1993).
(24) B. O. Fowler, M. Markovic, and W. E. Brown, “Octacalcium Phosphate 3. Infrared and Raman Vibrational Spectra,” Chem. Mater., 5, 1417-1423 (1993).
(25) C. Combes, C. Rey, and M. Freche, “in Vitro Crystallization of Octacalcium Phosphate on Type I Collagen: Influence of Serum Albumin,” J. Mater. Sci, Mater, M., 10, 153-180(1888).
(26) R. Tang and G. H. Nancollas, “Abnormal Dissolution of Octacaiciurn Phosphate Crystals at Constant Undersaturation,” J. Cryst. Growth, 212, 261-269 (2000).
(27) Y. H. Tseng, J. Zhan, K. S. K. Lin, C. Y. Mou, and J. C. C. Chan, “High Resolution ³¹P NMR Study of Ociacalciurn Phosphate,” Sol. Stale Nucl. Mag, Reson., 26, 99-104 (2004).
(28) O. Suzuki, S. Karnakura, T. Katagiri, M. Nakamura, B. Zhao,Y. Honda, and R.

Kamijo, “Bone Formation Enhanced by Implanted Octacalcium Phosphate involving Conversion into Ca-deficient Hydroxyapatite,” Biomaterials, 27, 2671-2681 (2006).
(29) R. M. Shelton, Y. Liu, P. R. Cooper, U. Gbureck, M. J. German, and J. E. Barraiet, “Bone Marrow Cell Gene Expression and Tissue Construct Assembly using Octacalcium Microscaffolds,” Biomaterials, 27, 2874-2881 (2006).
(30) Y. Liu, P, R. Cooper, J. E. Barralet, and R. M. Shelton, “Influence of Calcium Phosphate Crystal Assemblies on the Proliferation and Osteogenic Gene Expression of Rat Bone Marrow Stromal Cells,” Biomaterials, 28, 1393-1403 (2007).
(31) G. Falini, M. Gazzano, and A. Ripamonti, “Control of the Architectural Assembly of Octacalcium Phosphate Crystals in Denatured Collagenous Matrices,” J. Mater, Chem., 10, 535-538 (2000).
(32) S. Ban and J. Hasegawa, “Method for Producing Hexacalcium Phosphate,” JP 6122510 (A), Jun. 5, 1994.
(33) F. Monchau, A. Lefevre, M. Descamps, A. Belquin-Myrclycz, P. Laffargue, and H. F. Hildebrand, “In Vitro Studies of Human and Rat Osteoclast Activity on Hydroxyapatite, β-Tricalciurn Phosphate, Calcium Carbonate,” Biomol. Eng., 19, 143-152 (2002).

1. A method of preparing granules based on octacalcium phosphate (Ca₈(HPO₄)₂(PO₄)₄.5H₂O) for surgical use characterized in that the method comprises the steps of:
(a) preparing, at room temperature, an aqueous solution by using distilled water containing Na⁺, K⁺, Mg²⁺, Cl⁻, HCO₃⁻, NH₄⁺, H₂PO₄⁻, Ca²⁺ and/or HPO₄²⁻ ions,
(b) adding brushite (dicalcium phosphate dihydrate. CaHPO₄.2H₂O) granules into the solution,
(c) soaking the granules in the solution for 24 to 168 hours between 37° and 75° C. without stirring,
(d) separating the octacalciurn phosphate granules from the solution by filtration,
(e) washing the octacalcium phosphate granules with water,
(f) drying the octacalcium phosphate granules at 37° C. from 20 to 36 hours.

2. A method according to claim 1 characterized in that preparation method of said granules based on brushite (dicalcium phosphate dihydrate, CaHPO₄.2H₂O) for surgical use comprising the steps of:
(a) preparing, at room temperature, an aqueous solution by using distilled water containing H₂PO₄⁻ and/or HPO₄²⁻ ions,
(b) adding marble granules into the solution,
(c) soaking the granules in the solution for 12 to 24 hours at room temperature without stirring,
(d) separating the brushite granules from the solution by filtration,
(e) washing the brushite granules with water,
(f) drying the brushite granules at 37° C. from 20 to 36 hours.

3. The method according to claim 2 characterized in that the dihydrogen phosphate (H₂PO₄⁻) ion of the solution is supplied in the form of either sodium dihydrogen phosphate (NaH₂PO₄), potassium dihydrogen phosphate (KH₂PO₄), ammonium dihydrogen phosphate (NH₄H₂PO₄), or ortho-phosphoric acid (H₃PO₄).

4. The method according to claim 2 characterized in that the hydrogen phosphate (HPO₄²⁻) ion of the solution is supplied in the form of either disodium hydrogen phosphate (Na₂HPO₄), dipotassium hydrogen phosphate (K₂HPO₄) or diammonium hydrogen phosphate ((NH₄)₂HPO₄).

5. The method according to claim 2 characterized in that the pH value of the solution can be adjusted by adding small aliquots of ammonium hydroxide (NH₄OH) solution.

6. The method according to claim 2 characterized in that the concentration of the H₂PO₄⁻ ions present in the solution is between 1.5 and 2.5 molar.

7. The method according to claim 2 characterized in that the pH value of the solution is adjusted to about 4 at room temperature.

8. The method according to claim 2 characterized in that 2 to 40 grams of marble (calcium carbonate) granules are soaked in 50 to 1000 mL of solution at room temperature to produce brushite granules.

9. The method according to claim 1 characterized in that the said brushite granules have sizes in the range of 0.5 to 4 mm.

10. The method according to claim 2 characterized in that the obtained brushite granules are identified by X-ray diffraction data indicative of the brushite phase, directly collected over the granules.

11. The method according to claim 1 characterized in that the hydrogen phosphate (HPO₄²⁻) ion of the solution is supplied in the form of disodium hydrogen phosphate (Na₂HPO₄) over the solution concentration range of 1 to 4 millimolar.

12. The method according to claim 1 characterized in that the dihydrogen phosphate (H₂PO₄⁻) ion of the solution is supplied in the form of sodium dihydrogen phosphate (NaH₂PO₄) over the solution concentration range of 0.5 to 4 millimolar.

13. The method according to claim 1 characterized in that the potassium (K⁺ ion of the solution is supplied in the form of potassium chloride (KCl) over the solution concentration range of 4 to 6 millimolar.

14. The method according to claim 1 characterized in that the magnesium (Mg²⁺) ion of the solution is supplied in the form of magnesium chloride (MgCl₂) over the solution concentration range of 0.5 to 2.5 millimolar.

15. The method according to claim 1 characterized in that the calcium (Ca²⁺) ion of the solution is supplied in the form of calcium chloride (CaCl₂) over the solution concentration range of 0.5 to 25 miilimolar.

16. The method according to claim 1 characterized in that the bicarbonate (HCO₃⁻) ion of the solution is supplied in the form of sodium bicarbonate (NaHCO₃) over the solution concentration range of 4 to 65 millimolar.

17. The method according to claim 1 characterized in that the solution pH is adjusted at around 7.4.

18. The method according to claim 1 characterized in that the solution has a nominal Ca/P molar ratio between 1.667 and 2.

19. The method according to claim 1 characterized in that 0.35 to 4.5 grams of brushite granules are soaked in 75 to 1000 mL of solution to produce octacalciurn phosphate granules.

20. The method according to claim 1 characterized in that the obtained octacalcium phosphate granules are identified by X-ray diffraction data indicative of the octacalcium phosphate phase directly collected over the granules.

21. Brushite granules which are obtained by a method according to claim 2.

22. Octacalcium phosphate granules which are obtained by a method according to claim 1.

23. Biphasic mixtures of brushite-octacalcium phosphate granule mixtures which are obtained by first separately weighing and then physically bending the proper amounts of brushite and octacalcium phosphate granules produced by the methods according to claims 1 and 2.
